(19) United States
(12) Patent Application Publication
(10) Pub. No.: US 2026/0263138 A1
(43) Pub. Date: Sep. 10, 2026

(54) MEDICAL INSTRUMENT WITH BALANCED ELECTRODE CURRENTS

(71) Applicant: Erbe Elektromedizin GmbH, Tuebingen (DE)

(72) Inventor: Konstantin DORNHOF, Horb am Neckar (DE)

(21) Appl. No.: 19/555,328

(22) Filed: Mar. 3, 2026

(30) Foreign Application Priority Data
Mar. 7, 2025 (EP) 25162395.5

(51) Int. Cl. A61B 18/04; A61B 18/00

(52) U.S. Cl. CPC A61B 18/042; A61B 2018/00059; A61B 2018/00577; A61B 2018/00607

(57) ABSTRACT

For current supply of different electrodes of an instrument from a two-pole voltage source, for example a generator, a current balancing device serves that distributes the current supplied by the current source into two sub-currents. Preferably the current balancing device is an alternating current mirror circuit having two inductors (L1) and (L2) through which the current flows in opposite direction and which are arranged on a common magnetic circuit.

### CROSS-REFERENCE TO RELATED APPLICATION

This application claims priority to European Patent Application No. 25162395.5 filed Mar. 7, 2025, the entirety of which is incorporated herein.

### BACKGROUND AND SUMMARY

The invention refers to a medical instrument with balanced electrode currents, that means electrode currents that adapt with each other, for electrosurgical treatment of a human or animal patient.

Different instruments exist having multiple electrodes, from which a current for treatment of biological tissue originates respectively.

For example, EP 3 141 204 B1 and EP 3 141 203 B1 disclose respectively an instrument for large-scale mucosa ablation having a head that comprises two outlet openings with one electrode arranged therein in each case. The electrodes ionize the argon stream exiting the openings, so that multiple plasma jets exit the instrument. A generator producing a high-frequency high voltage is provided for current supply of the two electrodes. The high voltage is supplied via a switching arrangement to the electrodes. The switching arrangement connects the electrodes in quick sequence alternatingly with the generator output, so that a square wave modulated HF voltage with 50% ON-duration is applied to the two electrodes in each case. However, the current strengths of the two electrode currents are independent from each other.

For producing multiple plasma jets US 2005/0015086 A1 provides an instrument having multiple electrodes and plasma outlet openings. The electrodes are supplied from a generator via a switching unit. The switching unit serves to select a subgroup from the provided electrodes respectively and to connect it to the generator, so that from some openings of the instrument gas jet exit and from other openings plasma jets exit.

Further a coagulation-dissection instrument is known from EP 2 992 849 B1 that uses forceps-like basic configuration. The two jaws of the forceps-like instrument support sealing electrodes. In addition, one of the two jaws is equipped with a cutting electrode. A generator having a two-pole output serves to operate the instrument. While one of the output poles is connected with one of the coagulation electrodes, that then serves as neutral electrode, the other output pole of the generator is connected with the other sealing electrode of the other jaw. For current supply of the cutting electrode serves a transformer that can be arranged in the instrument and that serves to transform the voltage supplied by the two poles of the generator up to a higher value.

In all of the mentioned cases it is important to distribute the current supplied by the generator as desired on the electrodes of the instrument. In the case of the mentioned plasma instruments it is desired to supply both plasma or multiple plasma jets with equal current. In the case of the mentioned coagulation and dissection instruments it is desired to adjust the coagulation current and the cutting current in a manner corresponding to the purpose.

Starting therefrom it is the object of the invention to provide an instrument that comprises at least two electrodes that have to be supplied with current and in which a desired distribution of the current supplied by the generator to the electrodes is guaranteed.

This object is solved by means of embodiments of described instruments.

An example instrument according to the disclosure comprises two electrodes that are or can be connected via a current balancing device with only one first pole of a two-pole generator, the other, second pole of which is connected or can be connected with a neutral electrode. The current balancing device is configured to adjust a specific ratio of the two currents flowing to the two electrodes. For example, the current supplied by the generator can be equally distributed among two (or more) electrodes from which then plasma jets originate having substantially equal strengths. In this manner a uniform coagulation result on biological tissue can be obtained with instruments that produce multiple plasma jets.

The current balancing device preferably comprises an input which can be, or which is, connected with the first pole of the generator, and at least two outputs that are connected with the electrodes. In principle, it is possible to configure the current balancing device with active electronic components, such as according to the known current mirror circuit for direct currents. However, the current balancing device is preferably passive, that means configured without use of active components (for example semi-conductors). This applies particularly if the generator is a high-frequency generator (HF generator) that produces a high-frequency alternating voltage (HF voltage) and supplies a high-frequency current (HF current). The frequency of the current and the voltage is preferably between 100 kHz and 1 MHz, preferably at approximately 350 kHz. The current balancing device can also be considered and denoted as “HF current mirror circuit” or briefly as “HF current mirror”.

The passive current balancing device can comprise two inductors for mirroring the HF currents, which are arranged in magnetically coupled manner with each other, and which are thereby poled with opposite sense. The first inductor is arranged in the supply line to the first electrode and a first HF current flows through the first inductor, while the second inductor is arranged in the supply line to the second electrode and a second HF current flows through the second inductor. The inverse polarity of the inductors means that the inductors produce magnetic flows orientated in opposite direction in the common magnetic circuit during operational current flow. Due to the inverse polarity of the inductors, current symmetry results also in the case of different electrical loads on the two or multiple electrodes. For example, this can be the case with inclined orientation of the instrument due to different lengths of the plasma jets existing the two openings. Thereby it is particularly advantageous if the inductors arranged upstream of the electrodes comprise a coupling factor close to 1, however in any case larger than 0.9, preferably larger than 0.95.

For plasma instruments having two electrodes, frequently an equal distribution of 50% of the current to each electrode is desired for the current splitting. In this case it is advantageous if the inductors have equal number of turns. They can be wound onto a common core, particularly a ferrite ring core or another core geometry of magnetically soft material suitable for high frequencies. If three or more electrodes are provided, the current balancing device is configured to allocate a ratio of the current supplied by the generator to each electrode that is equal to 1 divided by the number of electrodes supplied with current.

It is also possible to provide different numbers of turns for the two inductors in order to balance the currents in another ratio than one to one.

Further details of embodiments of the invention disclosed herein and in the accompanying drawings.

### BRIEF DESCRIPTION OF DRAWINGS

FIG. 1 shows an instrument according to the invention connected to a supplying generator during use during a mucosa ablation,

FIG. 2 shows the distal end of the instrument according to FIG. 1 in a longitudinal cut illustration,

FIG. 3 shows a modified embodiment of the instrument according to FIG. 1 in a longitudinal cut illustration of its distal end,

FIG. 4 shows a schematic circuit illustration of an embodiment of the instrument with a current balancing device connected to a generator in accordance with disclosed embodiments,

FIG. 5 shows the current balancing device of FIG. 4 in accordance with disclosed embodiment,

FIG. 6 shows a coagulation and dissection instrument connected to a supplying generator in accordance with disclosed embodiments,

FIG. 7 shows the distal end of the instrument according to FIG. 6,

FIG. 8 shows an electrical schematic illustration of current balancing device for supply of electrodes of the instrument according to FIGS. 6 and 7 with unequal currents,

FIG. 9 shows an electrical schematic illustration of a current balancing device for supply of four electrodes in accordance with disclosed embodiments,

FIG. 10 shows an electrical schematic illustration of a current balancing device for two electrodes in a principal illustration in accordance with disclosed embodiments.

### DETAILED DESCRIPTION

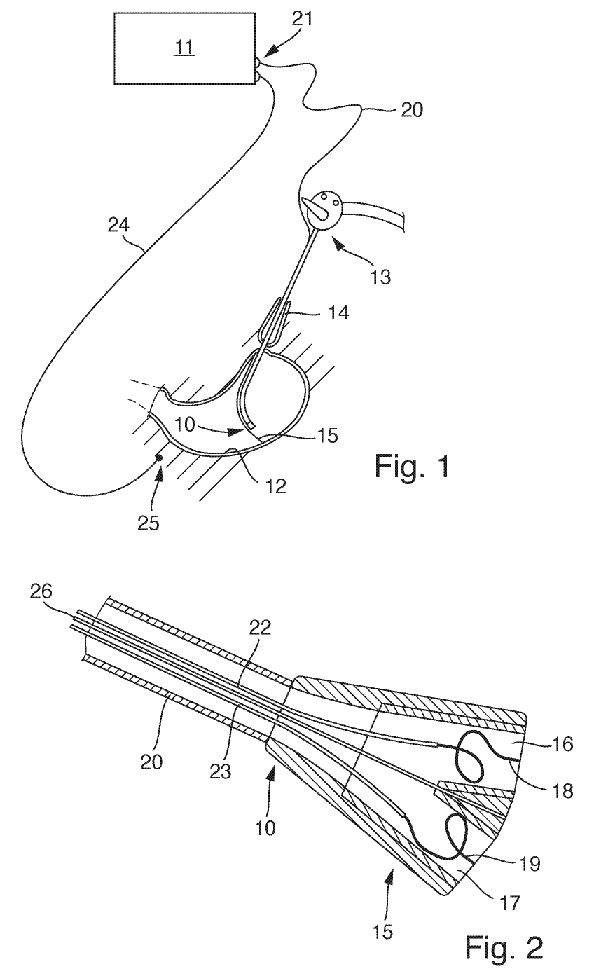

With reference to FIG. 1, an instrument 10 is shown configured as an argon plasma ablation probe and a supplying generator 11 that illustrated during endoscopic use during ablation of the gastric mucosa 12 of a patient(not further shown). The instrument 10 is inserted by means of the endoscope 13, for example through the esophagus 14 of the patient into the stomach and is guided there along the gastric mucosa 12 using the endoscope. On the distal end of the instrument 10, which is separately illustrated in FIG. 2, a head 15 is provided on which two gas outlet openings 16, 17 are provided with electrodes 18, 19 arranged therein. Alternatively, multiple electrodes can be provided that are arranged in respective gas outlet openings. It is further possible to arrange two or more electrodes in common, for example slit-shaped, gas outlet openings.

Independent from their number and shape, the gas outlet openings 16, 17 are supplied with gas, for example argon, via a hose 20. For this purpose, the hose 20 extends originating from head 15 up to the proximal end 21 (FIG. 1), where it is connected to a gas source using a suitable connecting means. The gas source can be arranged in the generator 11 provided for current supply to the electrodes 18, 19. Alternatively the gas source to which hose 20 is connected can be separately provided outside of generator 11.

The generator 11 serves to supply the two electrodes 18, 19 with high voltage, so that high frequency currents can originate from both electrodes 18, 19. These currents ionize the gas flow exiting the gas outlet openings 16, 17, so that head 15 ejects two plasma jets adjacent to one another. The electrodes 18, 19 are therefore connected with generator 11 via corresponding electrical lines 22, 23 that are insulated relative to each other. The generator 11 comprises (minimum) one oscillator for producing a high-frequency high voltage and for outputting high-frequency treatment currents. The lines 22, 23 extend from electrodes 18, 19 up to the proximal end 21 of hose 20 in order to be connected to the generator 11 there via the connecting means. A current balancing device 29 is arranged between the lines 22, 23 and the oscillator, wherein the current balancing device 29 is configured to equally distribute the current output by the oscillator onto the two electrodes 18, 19.

The generator 11 is connected with the tissue of the patient in low ohmic manner via another electrical line 24 and a neutral electrode 25, in order to pick up the treatment currents coming from the electrodes 18, 19 and to guide them back to the generator. The neutral electrode can be a two-dimensional adhesive electrode that is to be connected to a skin portion of the patient. It is configured to provide a two-dimensional electrical contact with the patient. The neutral electrode can however also be arranged on the instrument itself.

Sensor means can be provided on head 15, for example in the form of an optical fiber 26 that can be used for distance control, spectral analysis of the light created by the plasma jets or the like. Additional sensors are also possible.

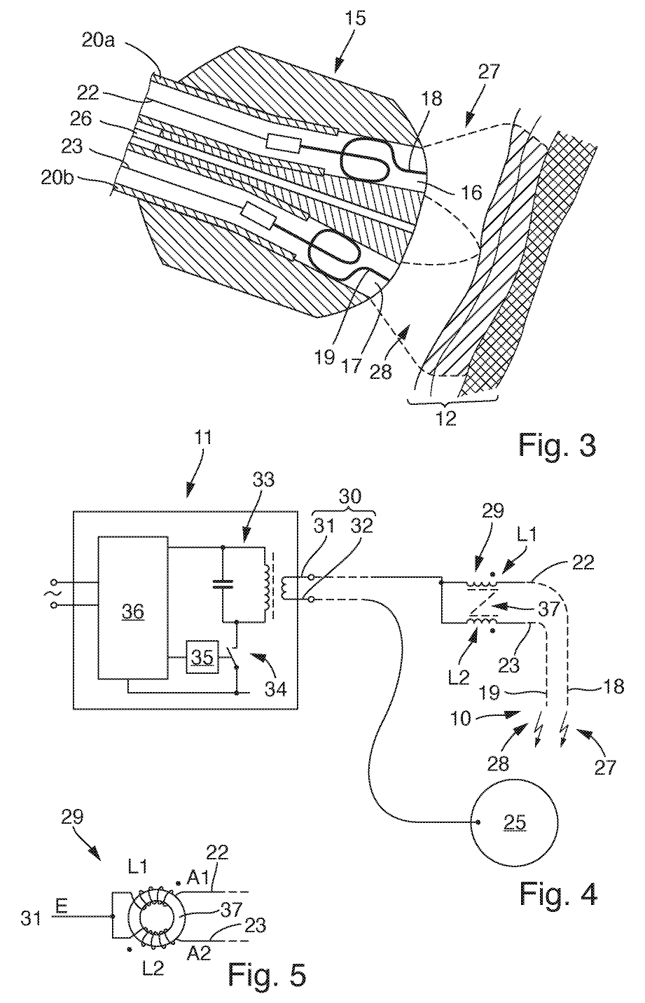

FIG. 3 illustrates a modified embodiment of the instrument 10 configured as an ablation probe with the aid of head 15. For the head 15 according to FIG. 3, the above description applies accordingly. In addition, it applies that instead of one single hose 20 for gas supply, two parallel guided hoses 20a and 20b are provided that are individually assigned to the two gas outlet openings 16, 17 individually.

From the two gas outlet openings 16, 17 plasma jets 27, 28 exit that impact on the gastric mucosa 12 and shall be substantially equally effective as far as possible. For this purpose, the instrument 10 and the generator 11 are configured so that the HF currents that transition from the electrodes 18, 19 into the argon gas have substantially equal amounts. For this purpose, the already mentioned current balancing device 29 is connected upstream of the electrodes 18, 19 of instrument 10 configured as an ablation probe, wherein the electrodes 18, 19 are connected with the generator 11 via the current balancing device 29. The current balancing device 29 is an HF current mirror circuit configured to equalize the HF currents flowing in the lines 22, 23 to one another. Thereby, the HF current supplied from generator 11 to the input E (FIG. 5) of the current balancing device is equally distributed on the two outputs A1, A2 to which the electrodes 18, 19 are connected. For example, the current balancing device 29 can be arranged inside a connector, which is arranged on the proximal end 21 of hose 20 and which serves to electrically connect the instrument 10 to generator 11.

For connection of the instrument 10, generator 11 typically comprises a two-pole output 30 (FIG. 4) comprising a first pole 31 for current supply of the electrodes 18, 19 and a second pole 32 for a connection of neutral electrode 25. Between the poles 31, 32, a high-frequency output voltage is applied that can be coupled out of an oscillating circuit 33 of generator 11 or that can be provided in another manner. For excitation of oscillating circuit 33, one or more electronic switches 34 may be included that can be opened and closed according to the clock of the high-frequency oscillation to be produced. A current supply unit 36, that is typically supplied by the grid, serves for energy supply.

The current flowing out of first pole 31 and the current flowing into second pole 32 are of equal amount. The current flowing out of pole 31 is divided by means of the current balancing device 29. Thereby the current balancing device 29 is configured to maintain the currents in the two lines 22 and 23 in an invariant ratio relative to one another, particularly in a ratio of 1:1.

The current flowing out of first pole 31 into the current balancing device 29 is a high-frequency alternating current, the frequency of which corresponds to the oscillating frequency of the parallel oscillating circuit 33. Similarly, the currents in the lines 22 and 23 are high-frequency alternating currents, the current strengths of which are half of the current strength of the current flowing out of pole 31 respectively.

In order to achieve an equalization of the currents in the lines 22, 23 and thus of the currents flowing out of the electrodes 18, 19 through the plasma jets 27, 28, the current balancing device 29 comprises a first inductor L1 connecting pole 31 with line 22. Additionally, current balancing device 29 comprises a second inductor L2 connecting pole 31 with line 23. Both inductors L1, L2 are arranged on a common magnetic circuit 37, wherein the inductors L1, L2 have inverse polarity. Due to the inverse polarity of inductors L1 and L2, the magnetic fields produced in the magnetic circuit 37 cancel each other due to the currents flowing therethrough respectively. For illustrating the term of “inverse polarity” of inductors L1 and L2, reference is made to FIG. 10. FIG. 10 shows for this purpose a schematic magnetic circuit illustration with identical topology as the current mirror circuit 29 according to FIGS. 4 or 5. The inductors L1 and L2 are inversely arranged in a sense, so that the inductors L1 and L2 produce oppositely orientated magnetic flows in the common magnetic circuit if they are passed by electrical currents having equal phases. With view from magnetic circuit 37 the magnetic flows produced by the two inductors are phase shifted about 180°.

The magnetic flows induced by the inductors cancel each other if the two currents have equal amounts (and are in phase). The inductors L1 and L2 have equal number of turns for this purpose. They are inductively coupled with each other via magnetic circuit 37, wherein the coupling factor of the two inductors L1, L2 with each other is preferably greater than 0.9, further preferably greater than 0.95 and is in the ideal case close to 1.0. In FIG. 4, the winding starting points of the inductors L1 and L2 are characterized using a dot. In this case the winding starting points are on the side of the electrodes 18, 19 in each case. In the practical configuration having a ring core and inductors L1, L2 wound with equal sense, the starting points of the windings of the inductors are opposite to each other, that means the winding starting point of inductor L1 is connected to the line 22, while the winding starting point of the inductor L2 is connected with pole 31.

The current balancing device 29 can be part of the generator 11 so that for connecting the lines 22, 23 individual sockets or contacts are provided respectively. Alternatively, the current balancing device 29 can be part of the instrument, that means the ablation probe 10, and can be arranged for this purpose, for example, inside the connector 21 thereof. It is further possible to configure the current balancing device 29 as a separate unit, for example as an intermediate connector, the input of which is to be connected to pole 31 and the two outputs of which is to be connected to the lines 22, 23.

The unit described so far that is configured with generator 11 and an instrument 10 configured as ablation probe, operates according to the following example:

It is assumed that the treating person has inserted the ablation probe into the patient, as illustrated in FIG. 1, and positioned it in proximity to the gastric mucosa 12. If now the ablation shall be started, first the gas source is activated so that a gas flow establishes in the hose 20 or in the hoses 20a, 20b and gas exits from the gas outlet openings 16, 17. Now, generator 11 is activated so that between the poles 31, 32 a high-frequency output voltage with multiple 100 up to over 1000 Volts is applied. Before electrical discharges form at the electrodes 18, 19 the gas path remains high ohmic, so that no voltage applies across the inductors l1, l2 and also no current flows therethrough.

If one of the electrodes 18, 19 ignites, for example first electrode 18, a plasma jet 27 exiting gas outlet opening 16 is produced, whereby the voltage at electrode 18 collapses down to lower values, typically below 100 Volt. The difference between this voltage and the voltage at first pole 31 is now applied to first inductor L1. The latter induces a voltage of equal amount in inductor L2 that is now added to the output voltage of generator 11 due to the inverse polarity of inductors L1 and L2, so that an increased (ignition) voltage applies to electrode 19 and therefore, practically immediately, a plasma discharge also forms at electrode 19. The two plasma jets 27, 28 are now burning, which can have different lengths due to irregularities of the gastric mucosa 12 as well as due to inclined orientation of head 15. Due to the magnetic coupling of the two inductors L1 and L2, the currents flowing in the lines 22, 23 equalize. Any current flowing through the inductor L1 induces a current of equal amount in second inductor L2. Similarly, any current flowing in inductor L2 induces a current of equal strength in inductor L1.

Due to the equalization of the current strengths in the currents supplied to the electrodes 18 and 19, it is achieved that the two plasma jets 27, 28 produce coagulation effects on the mucosa 12 of equal strength. In this manner the treating person can achieve uniform coagulation results without the need to watch out for maintaining absolutely equal distances between the gas outlet openings 16, 17 and the gastric mucosa 12.

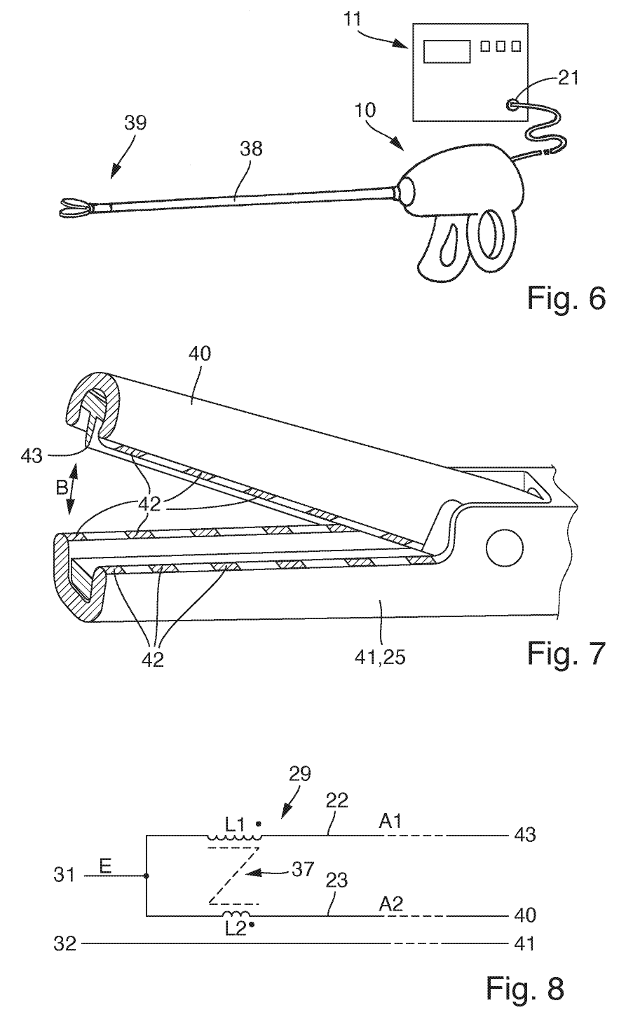

The present invention is not only suitable for current alignment of electrodes having similar functions, as it is the case in the instrument 10 configured as ablation probe, but is also suitable for current alignment in instruments with electrodes that have different functions. This will be explained subsequently based on FIGS. 6 to 8.

The instrument 10 illustrated in FIG. 6 is a laparoscopic forceps instrument on the shank 38 of which a coagulation and dissection tool 39 is supported. The latter comprises two jaws that are moveably supported in a manner to be pivotable toward and away from one another and that have electrodes serving for tissue coagulation. Thereby, the jaws 40, 41 can have insulated locations 42 on the coagulation surfaces facing each other, in order to avoid electrical short circuits between the electrodes when closing the jaws.

As depicted in FIG. 8, the jaw 41 or the electrode arranged thereon or formed by the latter can be considered as neutral electrode connected with second pole 32 of generator 11.

In the other jaw 40 a cutting electrode 43 can be immovably arranged serving to separate tissue held between the jaws 40, 41 and coagulated due to current supply. Thereby, it can be necessary that the current supplied to the coagulation electrode of jaw 40 is in a specific ratio compared with the current supplied to cutting electrode 43. In this case in the housing of instrument 10 or also at another position (for example inside instrument connector) the current balancing device 29 according to FIG. 8 can be arranged.

The current balancing device 29 comprises, as described above, two inductors L1 and L2 coupled with each other via magnetic circuit 37. The coagulation electrode of jaw 40 is supplied with current via inductor L2. The cutting electrode 43 is supplied with current via inductor L1. In this case the inductances of inductors L1, L2 and thus the number of turns thereof can be defined differently, for example in order to define the cutting current at cutting electrode 43 in a specific ratio compared to coagulation current at jaw 40.

Again, the coupling factor is preferably greater than 0.90, better 0.95 and in the best case close to 1.0. In addition, it can be provided that one of the two inductors L1, L2 (or also both) can be bridged by an arbitrarily operable switch in order to make the inductivity of the inductor L1 and L2 ineffective. Independent therefrom it can be provided to provide a switch for interrupting the current in one of the two inductors L1 or L2 in order to be able to specifically deactivate the cutting electrode 43 and/or the coagulation electrode provided at the jaw 40.

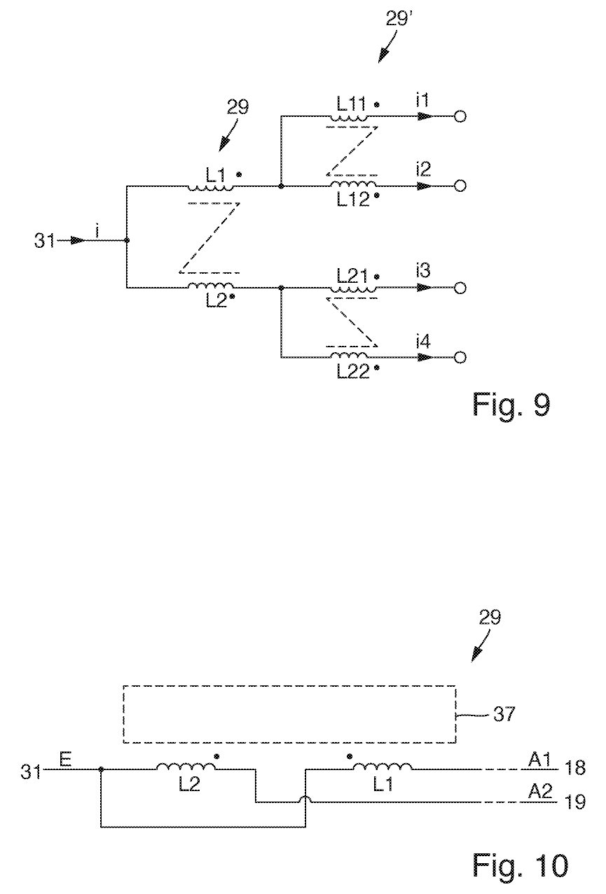

FIG. 9 illustrates the configuration of a current balancing device 29' serving for splitting a current i into four sub-currents i1 to i4 having equal amounts. Thereby the inductors L1, L2 of first current balancing device 29 have coinciding values. Also, the inductivities of the following inductors L11 and L12 coincide with each other; just like the inductors L21, L22. When selecting inductivities of different amount, the ratios of the currents i1 to i4 compared to each other can be adjusted in a manner deviating from a ratio of 1:1. If an odd number of electrodes shall be supplied, the first current balancing device 29 can be configured asymmetrically and split the currents in a ratio 1:2. The stronger of the split currents can be split using another current balancing device 29 in a ratio of 1:1. In this manner a current distribution of the three currents of 1/3 : 1/3 : 1/3 is obtained.

For current supply of different electrodes 18, 19 of an instrument 10 from a two-pole voltage source, for example a generator 11, a current balancing device 29 is described that distributes the current supplied by the current source into two sub-currents. Preferably the current balancing device 29 is an alternating current mirror circuit having two inductors L1 and L2 through which the current flows in opposite direction and which are arranged on a common magnetic circuit 37.

1. An instrument for medical treatment of biological tissue, the instrument comprising:
at least a first electrode and a second electrode adapted to be connected or are connected via a current balancing device with only one first pole of a generator having a two-pole output whose other, second pole is connected or is adapted to be connected with a neutral electrode.

2. The instrument according to claim 1, wherein the current balancing device comprises an input adapted to be connected or connected with the first pole and at least two outputs , which are respectively connected with one of the at least first and second electrodes.

3. The instrument according to claim 1, wherein the current balancing device comprises a first inductor and a second inductor that are magnetically coupled with each other and that have inverse polarity in that the first inductor and the second inductor are arranged in a manner producing inverse fields in a common magnetic circuit.

4. The instrument according to claim 1, wherein the first and second inductors are wound in a bifilar manner.

5. The instrument according to claim 3, wherein the first and second inductors have a coupling factor (K) that is greater than 0.9, preferably greater than 0.95.

6. The instrument according to claim 3, wherein the first and second inductors comprise a number of turns that coincide with each other.

7. The instrument according to claim 3, wherein the at least first and second electrodes are identically configured.

8. The instrument according to claim 1, wherein the first and second inductors have a number of turns that are different than one another.

9. The instrument according to claim 1, wherein the at least first and second electrodes are configured in a different manner than one another.

10. The instrument according to claim 1, further comprising one or multiple gas outlet openings that are arranged at a distal end of the instrument.

11. The instrument according to claim 10, wherein the one or multiple gas outlet openings are adapted to be supplied with a gas via a gas channel of the instrument for producing a plasma jet.

12. The instrument according to claim 11, wherein the gas channel is configured in a manner extending from the distal end of the instrument up to a proximal end of the instrument.

13. The instrument according to claim 12, wherein the instrument (comprises a connection means at its proximal end for connecting the gas channel to a gas supply source.

14. The instrument according to claim 2, wherein the instrument comprises a connection means at its proximal end for connecting the input of the current balancing device to the generator.

15. The instrument according to claim 10, wherein the at least first and second electrodes are arranged inside the one or the multiple gas outlet openings.
